(19) United States
(12) Patent Application Publication
(10) Pub. No.: US 2016/0206846 A1
(43) Pub. Date: Jul. 21, 2016

(54) TRACHEOSTOMA DEVICE MAGNETIC ATTACHMENT METHOD AND APPARATUS

(71) Applicant: Freudenberg Medical, LLC, Carpinteria, CA (US)

(72) Inventor: Eric D. Blom, Carmel, IN (US)

(21) Appl. No.: 14/993,062

(22) Filed: Jan. 11, 2016

Related U.S. Application Data
(60) Provisional application No. 62/103,849, filed on Jan. 15, 2015.

(51) Int. Cl. A61M 16/10; A61M 16/08; A61M 16/04

(52) U.S. Cl. CPC A61M 16/1045; A61M 16/0468; A61M 16/08

(57) ABSTRACT

A collar constructed from or including a ferromagnetic material snaps into position around the inner end of a heat and moisture exchanger (HME) or a hands-free tracheostoma valve for mounting on the end of a laryngectomy tube or neck-mounted housing, and a magnet on the outermost region of the laryngectomy tube or neck-mounted housing for attracting the collar. The collar includes a central opening through which air and respiration products flowing through the HME or hands-free tracheostoma valve may pass. The HME or hands-free tracheostoma valve is readily attached to the laryngectomy tube or neck-mounted housing by bringing the HME or hands-free tracheostoma valve with the collar fitted thereto into close proximity to the magnet.

### CROSS-REFERENCE TO RELATED APPLICATIONS

This application claims the benefit of U.S. Provisional Application No. 62/103,849, filed on Jan. 15, 2015. The entire disclosure of the above application is incorporated herein by reference.

### FIELD

The present disclosure relates to a heat and moisture exchange and hands free tracheostoma valve and more particularly to a magnetic attachment method and apparatus for a tracheostoma valve.

### BACKGROUND

Tracheostoma devices such as heat and moisture exchangers (hereinafter sometimes HMEs) and hands-free tracheostoma valves, and uses thereof, are known. See, for example, U.S. Pat. Nos. 4,582,058; 7,025,784; and 7,370,654 and references cited therein. The disclosures of these references are hereby incorporated herein by reference. This listing is not intended as a representation that a complete search of all relevant prior art has been conducted, or that no better references than those listed exist.

Many patients who wear such tracheostoma devices have relatively more limited dexterity, and relatively less strength in their hands. This makes it more difficult for such a patient to snap the HME and/or hands-free valve into a laryngectomy tube or neck-mounted housing (sometimes called a tape or adhesive housing) worn by the patient. Such snap mountings are typically rib-and-groove engagements between the tracheostoma device housing on the one hand, and the outer end of the tracheostoma base such as a laryngectomy tube, button or an adhesive housing on the other.

### SUMMARY

According to an aspect of the present disclosure, a combination includes a tracheostoma device having a cup constructed from, or containing a suitable amount of, a ferromagnetic material for positioning around the inner end of a heat and moisture exchanger (HME) or a hands-free tracheostoma valve for mounting on the end of a tracheostoma base such as a laryngectomy tube or neck-mounted housing and a magnet on the outermost region of the laryngectomy tube or neck-mounted housing for attracting the cup to attach the tracheostoma device to the end of the tracheostoma base.

Illustratively according to this aspect, the tracheostoma base in the form of a laryngectomy tube or neck-mounted housing includes a central passageway for the flow of air and respiration products through the tracheostoma device in the form of a HME or hands-free tracheostoma valve. The magnet surrounds at least a portion of the central passageway.

Illustratively according to this aspect, the magnet is molded into or attached by a suitable, relatively permanent adhesive to the outermost region of the tracheostoma base laryngectomy tube or neck-mounted housing.

According to another aspect, a combination includes a cup containing a ferromagnetic material for positioning around the inner end of a hands-free tracheostoma valve for mounting on the end of a tracheostoma base in the form of a laryngectomy tube or neck-mounted housing and a magnet on the outermost region of the laryngectomy tube or neck-mounted housing for attracting the cup to attach the hands-free tracheostoma valve to the end of the laryngectomy tube or neck-mounted housing.

Illustratively according to this aspect, the laryngectomy tube or neck-mounted housing includes a central passageway for the flow of air and respiration products through the hands-free tracheostoma valve. The magnet surrounds at least a portion of the central passageway.

Illustratively according to this aspect, the magnet is molded into or attached by a suitable, relatively permanent adhesive to the outermost region of the laryngectomy tube or neck-mounted housing.

According to another aspect, a combination includes a cup containing a ferromagnetic material for positioning around the inner end of a heat and moisture exchanger (HME) for mounting on the end of a tracheostoma base in the form of a laryngectomy tube or neck-mounted housing and a magnet on the outermost region of the tracheostoma base for attracting the cup to attach the HME to the end of the tracheostoma base.

Illustratively according to this aspect, the tracheostoma base includes a central passageway for the flow of air and respiration products through the HME, the magnet surrounding at least a portion of the central passageway.

Illustratively according to this aspect, the magnet is molded into or attached by a suitable, relatively permanent adhesive to the outermost region of the laryngectomy tube or neck-mounted housing.

### DRAWINGS

The drawings described herein are for illustrative purposes only of selected embodiments and not all possible implementations, and are not intended to limit the scope of the present disclosure. The detailed description particularly refers to the following figures, in which:

FIG. 1 illustrates an exploded perspective view of a laryngectomy tube and a heat and moisture exchanger (HME) device and a hands-free tracheostoma valve device for mounting on an end of the laryngectomy tube;

FIG. 2 illustrates an exploded perspective of the HME device of FIG. 1 showing the HME and a mounting collar;

FIG. 3 illustrates the HME device of FIGS. 1 and 2 mounted on the end of the laryngectomy tube;

FIG. 4 illustrates an exploded perspective view of a tracheostoma button and a heat and moisture exchanger (HME) device and a hands-free tracheostoma valve device for mounting on an end of a tracheostoma button;

FIG. 5 illustrates an exploded perspective view of a neck-mounted housing and a heat and moisture exchanger (HME) device and a hands-free tracheostoma valve device for mounting on an end of a neck-mounted housing; and

FIG. 6 illustrates an exploded perspective view of a heat and moisture exchanger (HME) device with an external metal ring or magnet ring with an alternative laryngectomy tube and neck mounted housing.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the disclosure as defined by the appended claims.

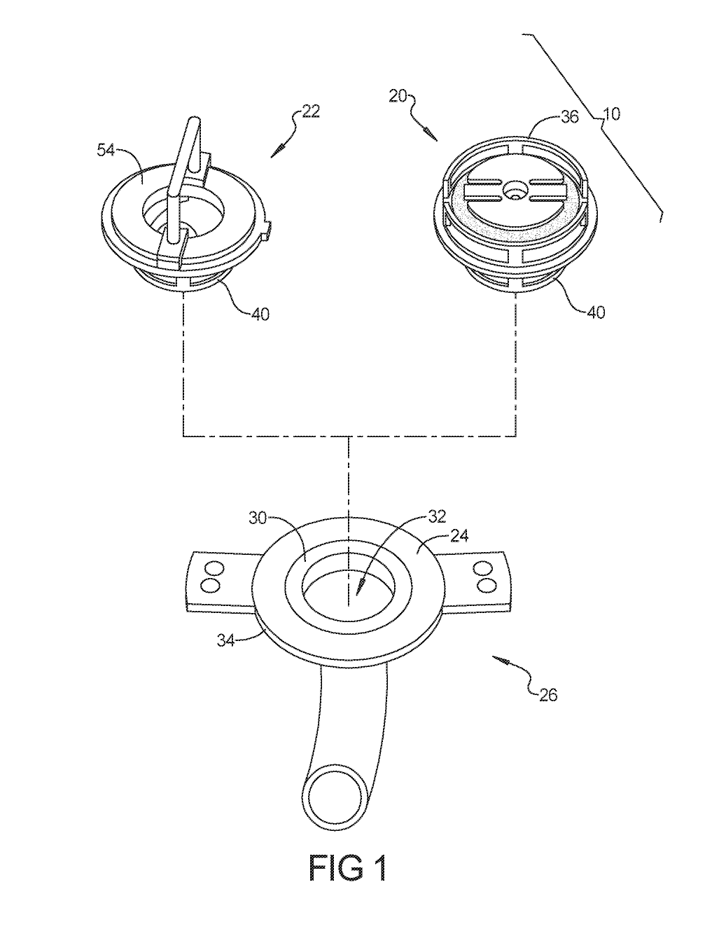

Referring now to FIG. 1, a prosthesis system 10 is illustrated. The system 10 includes a heat and moisture exchanger (HME) device 20 and a hands-free tracheostoma valve device 22 configured to be separately mounted on an end 24 of a laryngectomy tube 26. The laryngectomy tube 26 includes a central passageway 32 for the flow of air and respiration products through the device 20 or 22 when the device 20 or 22 is mounted on the tube end 24. The end 24 of the laryngectomy tube 26 includes (a) magnet(s) 30, illustratively in the shape of a ring, although other shapes and arrangements of the magnet(s) 30 can be used including one or magnet segments. The magnet 30 is molded into or attached by a suitable, press-fit, mechanical connection or relatively permanent adhesive to the outermost region/flange 34 (that is, the region 34 lying outside the neck of the wearer when in use orientation in/on the wearer) of the laryngectomy tube 26.

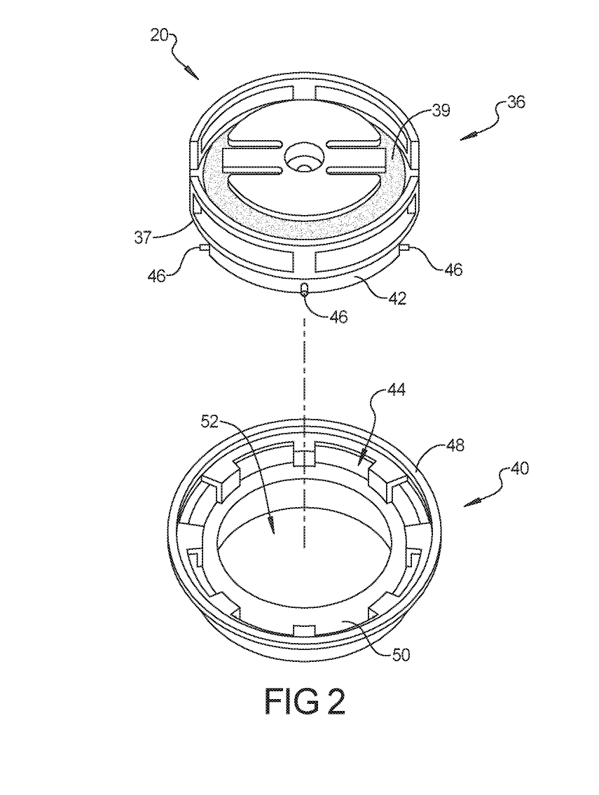

The HME device 20 includes a heat and moisture exchanger 36 and a housing structure that can include a mounting collar 40 that is secured to the HME 36. One example of an HME device 20 is the Blom-Singer HME, which is commercially available from Inhealth Technologies. As illustrated in FIG. 2, the HME 36 includes a mounting structure 37 with an inner end 42 sized to be positioned in a passageway 44 defined in the collar 40. A foam-like filter 39 can be supported by the mounting structure 37. Filter 39 retains moisture from exhaled air and transfers moisture to inhaled air passing through the HME device 20. In the illustrative embodiment, the collar 40 is secured to the HME 36 via a number of ribs 46 of the mounting structure 37 that snap into engagement with the annular rim 48 of the collar 40 when the inner end 42 of the HME 36 is positioned in the passageway 44. It should be appreciated that in other embodiments other combinations of tabs, grooves, or other fasteners may be used to secure the HME 36 to the mounting collar 40.

The mounting collar 40 can be constructed from, or contain, a ferromagnetic material to permit the collar 40 to be secured to the laryngectomy tube 26. In the illustrative embodiment, the ferromagnetic material takes the form of a metallic ring 50 positioned in the collar 40 at the end of the passageway 44 opposite the annular rim 48. The ring 50 includes a central opening 52 through which air and respiration products flowing through the device 20 or 26 may pass. Although the metallic ring is shown as a ring, other shapes or arrangements of one or more metallic segments can be used. The metallic component 50 can also be magnetic.

Returning to FIG. 1, the hands-free tracheostoma valve device 22 includes a hands-free tracheostoma valve member 54 and a housing structure that can include another mounting collar 40 that is secured to the valve 54. One example of a hands-free tracheostoma valve is the Blom-Singer Adjustable Tracheostoma Valve II (ATSV II), which is commercially available from Inhealth Technologies. The valve member 54, like the HME 36, includes an inner end that is sized to be positioned in the passageway 44 defined in the collar 40, and a number of ribs snap into engagement with the annular rim 48 of the collar 40 when the inner end of the valve member 54 is positioned in the passageway 44, thereby securing the valve member 54 to the collar 40.

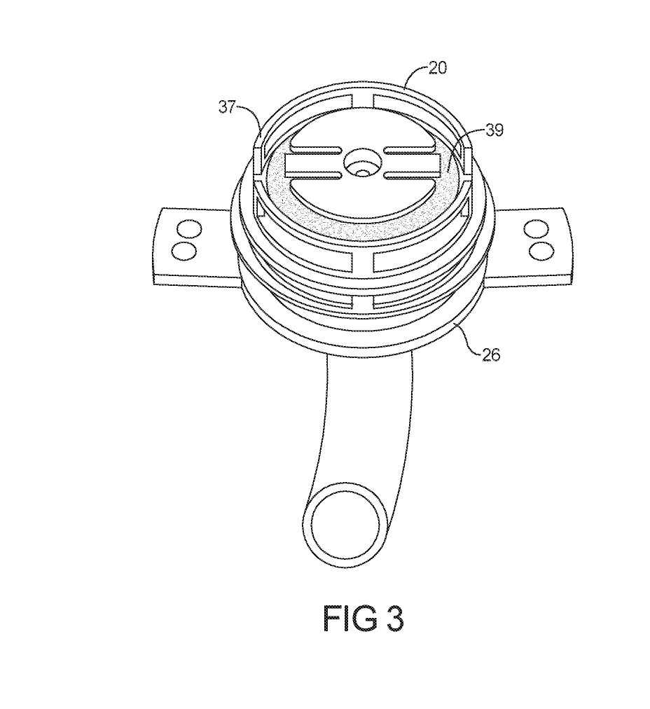

The devices 20 or 22 may readily be attached to laryngectomy tube 26 by bringing the device 20 or 22 into close proximity to the magnet 30 on the tube end 24. The configurations of magnet 30 and mounting cup 40 insure that device 20 or 26 will be appropriately oriented on laryngectomy tube 26 to support the flow of air and respiration products through the device 20 or 22 according to device 20's or 22's design parameters. For example, as illustrated in FIG. 3, the HME device 20 is mounted to the tube 26 such that the passageway 32 of the tube 26 is aligned with the central opening 52 of the collar 40 such that air and respiration products may flow through the passageway 32 into the central opening 52 and into the HME. While the ferromagnetic connection between the collar 40 and magnet 30 is sufficiently robust to maintain the device 20 or 22 on the laryngectomy tube 26 during normal wear, the device 20 or 22 is not held on so tightly that it cannot be snapped off the laryngectomy tube 26 with a little effort by the sometimes impaired strength- and/or dexterity wearer.

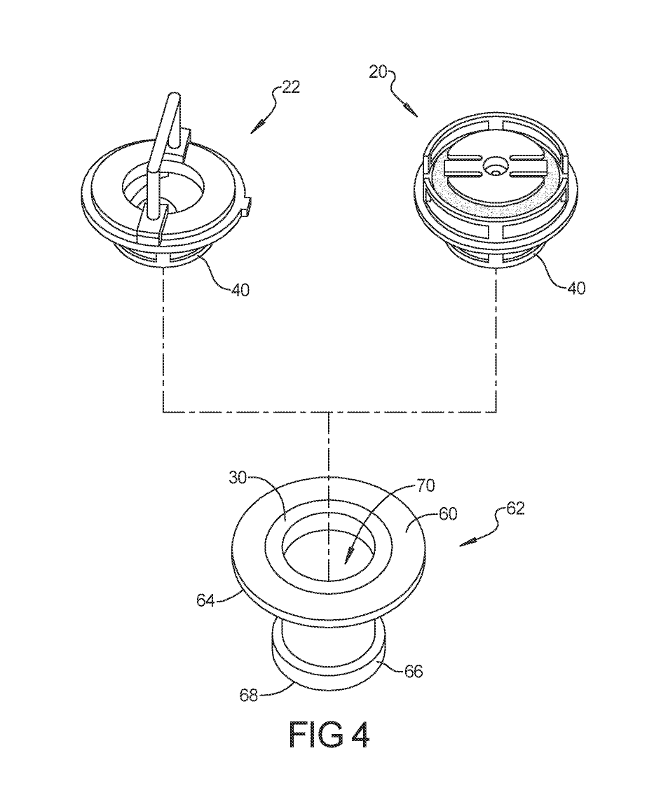

Referring now to FIG. 4, the HME device 20 and the hands-free tracheostoma valve device 22 are also configured to be separately mounted on an end 60 of a tracheostoma button 62. The button 62 includes a flange 64 at the exterior end 60 and another flange 66 at the opposite interior end 68. A passageway 70 extends between the ends 60, 68 of the button 62 for the air and respiration products to flow through the device 20 or 22 when the device 20 or 22 is mounted on the end 60. The end 60 of the tracheostoma button 62 includes (a) magnet(s) 30, illustrated in the shape of a ring. The magnet 30 is molded into or attached by a suitable, press-fit, mechanical connection or relatively permanent adhesive to the flange 64 of the tracheostoma button 62, which positions the magnet 30 outside the neck of the wearer when the button 62 is in use orientation in/on the wearer.

The devices 20 or 22 may readily be attached to tracheostoma button 62 by bringing the device 20 or 22 into close proximity to the magnet 30 on the button end 60. The configurations of magnet 30 and mounting cup 40 insure that device 20 or 26 will be appropriately oriented on tracheostoma button 62 to support the flow of air and respiration products through the device 20 or 22 according to device 20's or 22's design parameters. For example, the HME device 20 snaps onto the button 62 such that the passageway 70 of the button 62 is aligned with the central opening 52 of the cup 40 so that air and respiration products may flow through the passageway 70, the central opening 52 and the HME 20. While the ferromagnetic connection between the cup 40 and the magnet 30 is sufficiently robust to maintain the device 20 or 22 on the button 62 during normal wear, the device 20 or 22 is not held on so tightly that it cannot be snapped off the button 62 or with a little effort by the sometimes impaired strength- and/or dexterity wearer.

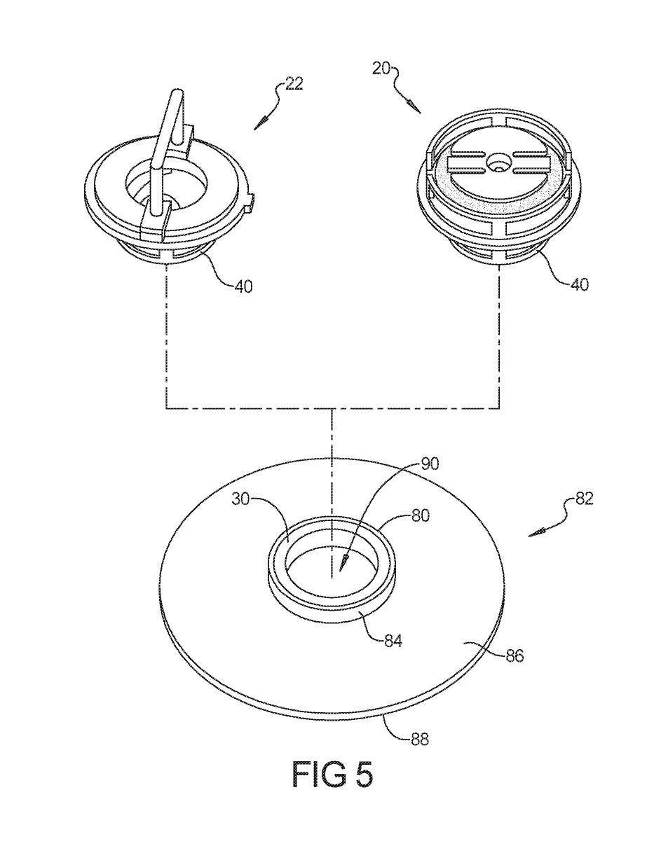

Referring now to FIG. 5, the HME device 20 and the hands-free tracheostoma valve device 22 are also configured to be separately mounted on an end 80 of an adhesive housing 82. The adhesive housing 82 includes a sleeve 84 positioned at the end 80 and a skirt 86 positioned at the opposite end 88. A passageway 90 extends between ends 80, 88 of the housing 82 for the flow of air and respiration products through the device 20 or 22 when the device 20 or 22 is mounted on the end 80. The end 80 of the adhesive housing 82 includes (a) magnet(s) 30, which is illustrated in the shape of a ring. The magnet 30 is molded into or attached by a suitable, press-fit, mechanical engagement or relatively permanent adhesive to the sleeve 84 of the adhesive housing 82, which is positioned outside the neck of the wearer when the adhesive housing 82 is in use orientation in/on the wearer.

The devices 20 or 22 may readily be attached to adhesive housing 82 by bringing the device 20 or 22 into close proximity to the magnet 30 on the housing end 80. The configurations of magnet 30 and mounting collar 40 insure that device 20 or 26 will be appropriately oriented on adhesive housing 82 to support the flow of air and respiration products through the device 20 or 22 according to device 20's or 22's design parameters. For example, the HME device 20 is mounted to the adhesive housing 82 such that the passageway 90 of the housing 82 is aligned with the central opening 52 of the collar 40 so that air and respiration products may flow through the passageway 90, the central opening 52 and the HME 20. While the ferromagnetic connection between the cup 40 and the magnet 30 is sufficiently robust to maintain the device 20 or 22 on the adhesive housing 82 during normal wear, the device 20 or 22 is not held on so tightly that it cannot be snapped off the adhesive housing 82 with a little effort by the sometimes impaired strength- and/or dexterity wearer.

While the disclosure has been illustrated and described in detail in the drawings and foregoing description, such an illustration and description is to be considered as exemplary and not restrictive in character, it being understood that only illustrative embodiments have been illustrated and described and that all changes and modifications that come within the spirit of the disclosure are desired to be protected. It should be understood that although the magnet 30 has been disclosed as part of the tracheostoma base in the form of a tracheostoma tube 26, button 62 or the adhesive housing 82 and the metallic ring 50 has been disclosed as being on the tracheostoma device 20, 22, the locations of the magnetic ring 30 and metallic ring 50 could be reversed. In addition, the metallic ring 50 can also be magnetized so that a magnetic ring can be used on both the tracheostoma device 20, 22 and the tracheostoma base 26, 62, 82.

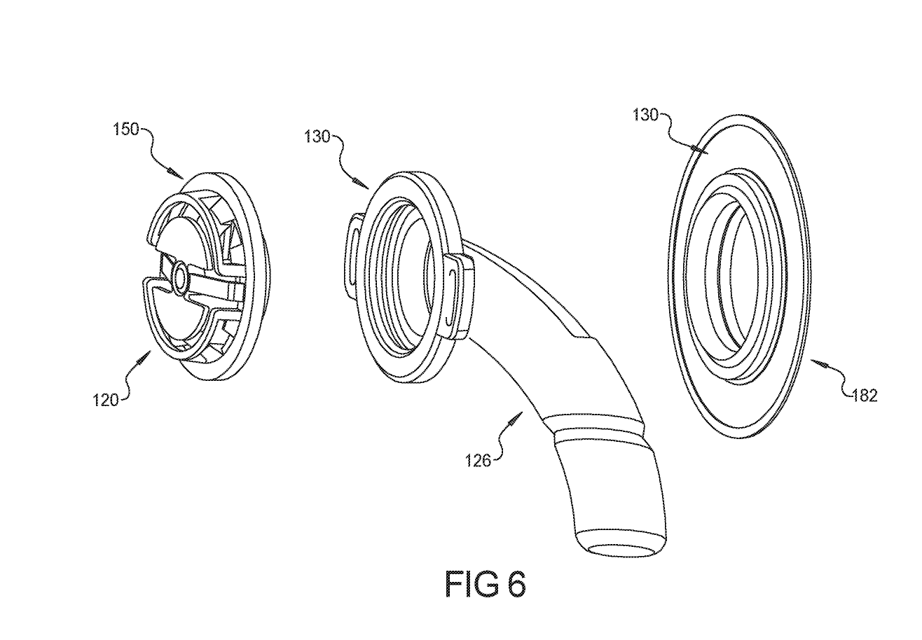

In the HME device 20 and the tracheostoma valve device 22 described above, the metallic or magnetic ring 30 50 is shown contained within the housing structure. With reference to FIG. 6, the HME cartridge 120 can be provided with an exterior metal or magnetic ring 150 and the laryngectomy tube 126 and/or adhesive housing 182 can be provided with a magnet or metal ring 130. The metal or magnetic rings 130, 150 can be molded into or attached by a suitable, press-fit, mechanical engagement or relatively permanent adhesive to the housing structure of the HME cartridge 120, laryngectomy tube 126 and the adhesive housing 182. A metal ring or magnetic ring 150 can also be similarly mounted to an exterior of the housing structure of the tracheostoma valve device 22.

There is a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

What is claimed is:

1. A mounting arrangement for a tracheostoma device, comprising:
a tracheostoma base having a central passageway and including one of a ferromagnetic material and a magnet associated there with;
a tracheostoma device including a housing structure for mounting engagement with the tracheostoma base and including the other of a ferromagnetic material and a magnet wherein the magnet attracts the ferromagnetic material to secure the tracheostoma device to the tracheostoma base.

2. The mounting arrangement according to claim 1, wherein the tracheostoma device is a heat and moisture exchanger.

3. The mounting arrangement according to claim 1, wherein the tracheostoma device is a hands-free tracheostoma valve.

4. The mounting arrangement according to claim 1, wherein the tracheostoma base is a laryngectomy tube.

5. The mounting arrangement according to claim 1, wherein the tracheostoma base is a tracheostoma button.

6. The mounting arrangement according to claim 1, wherein the tracheostoma base is an adhesive housing.

7. The mounting arrangement according to claim 1, wherein the ferromagnetic material is a magnet.

8. The mounting arrangement according to claim 1, wherein the ferromagnetic material and the magnet are ring-shaped.

9. The mounting arrangement according to claim 8, wherein the other of a ferromagnetic material and a magnet of the tracheostoma device is ring-shaped and mounted to an outer surface of the housing of the tracheostoma device.

10. A mounting arrangement for a tracheostoma device, comprising:
a tracheostoma base having a central passageway and including one of a ferromagnetic material and a magnet associated there with;
a heat and moisture exchanger including a housing structure for mounting engagement with the tracheostoma base and including the other of a ferromagnetic material and a magnet wherein the magnet attracts the ferromagnetic material to secure the heat and moisture exchanger to the tracheostoma base.

11. The mounting arrangement according to claim 10, wherein the tracheostoma base is a laryngectomy tube.

12. The mounting arrangement according to claim 10, wherein the tracheostoma base is a tracheostoma button.

13. The mounting arrangement according to claim 10, wherein the tracheostoma base is an adhesive housing.

14. The mounting arrangement according to claim 10, wherein the ferromagnetic material is a magnet.

15. The mounting arrangement according to claim 10, wherein the other of a ferromagnetic material and a magnet of the heat and moisture exchanger is ring-shaped and mounted to an outer surface of the housing structure of the heat and moisture exchanger.

16. A mounting arrangement for a tracheostoma device, comprising:
a tracheostoma base having a central passageway and including one of a ferromagnetic material and a magnet associated there with;
a tracheostoma valve including a housing structure for mounting engagement with the tracheostoma base and including the other of a ferromagnetic material and a magnet wherein the magnet attracts the ferromagnetic material to secure the tracheostoma valve to the tracheostoma base.

17. The mounting arrangement according to claim 16, wherein the tracheostoma base is a laryngectomy tube.

18. The mounting arrangement according to claim 16, wherein the tracheostoma base is a tracheostoma button.

19. The mounting arrangement according to claim 16, wherein the tracheostoma base is an adhesive housing.

20. The mounting arrangement according to claim 16, wherein the ferromagnetic material is a magnet.
